(19) United States
(12) Patent Application Publication
(10) Pub. No.: US 2019/0008832 A1
(43) Pub. Date: Jan. 10, 2019

(54) COMPOSITIONS FOR THE TREATMENT OF PRESBYOPIA

(71) Applicant: Roberto PINELLI, Paradiso (CH)

(72) Inventor: Roberto PINELLI, Paradiso (CH)

(21) Appl. No.: 16/067,247

(22) PCT Filed: Dec. 22, 2016
(86) PCT No.: PCT/IB2016/057917

§ 371 Date: Jun. 29, 2018

(30) Foreign Application Priority Data
Dec. 29, 2015 (CH) 01917/15

(51) Int. Cl. A61K 31/4178; A61P 27/10; A61K 9/00

(52) U.S. Cl. CPC A61K 31/4178; A61P 27/10; A61K 45/06; A61K 2300/00; A61K 9/0048

(57) ABSTRACT

The present invention relates to a composition comprising (a) pilocarpine or pharmaceutically acceptable salts thereof, (b) at least one alpha-stimulant agonist or pharmaceutically acceptable salts thereof and/or (c) at least one nonsteroidal anti-inflammatory agent (NSAID) or pharmaceutically acceptable salts thereof wherein (a) is present in a percentage by weight lower than 0.40%, (b) and/or (c) is present in a percentage by weight lower than 0.090% based on the total volume of the composition.

### FIELD OF THE INVENTION

The present invention relates to a composition comprising an agonist of the muscarinic acetylcholine receptor M3 in a percentage lower than 0.40% by weight on the total volume of the composition; its use in the treatment of presbyopia is a further object of the invention.

### BACKGROUND ART

Presbyopia is a physiological condition of the visual system that consists of a decrease in the power of accommodation of the eye caused by a progressive stiffening of the crystalline lens and/or by the weakening of the muscle that regulates accommodation.

Generally, presbyopia appears around 45 years of age in subjects who have no visual impairments (so-called emmetropes), whereas in people affected by hypermetropia the onset of presbyopia is early and has, as a side effect, an almost simultaneous difficulty in accommodation from a distance; in subjects with myopia, the phenomenon of presbyopia instead generally occurs later on. Presbyopia usually stabilizes around 65 years of age.

In young people, the crystalline lens is soft and flexible, ready to change shape in response to the contraction of the ciliary muscle of the eye to see images from different distances. With age, the crystalline lens tends to stiffen, losing elasticity. With loss of elasticity, the eye no longer has the same ability to shape itself correctly in order to focus nearby objects, at a distance of 40 centimeters or less from the eye.

In the eye, the ciliary muscle is under the control of the parasympathetic nervous system through acetylcholine and its muscarinic receptors. The sympathetic nervous system has a secondary regulatory role on the ciliary muscle by means of its alpha and beta receptors. Agonists or muscarinic stimulants and stimulants of alpha-2 and beta-2 receptors increase the contraction of the ciliary muscle and therefore may be able to obviate the stiffness of the crystalline lens caused by age, for as long as the stimulation is effective.

The sphincter of the iris, too, is mainly under the control of the parasympathetic system through muscarinic receptors, but it has no alpha and beta receptors; the dilator muscle of the iris is under the control of the sympathetic system via the alpha-1 and alpha-2 receptors. Alpha-1 stimulants cause dilation and alpha-2 stimulants limit dilation. The depth of the visual field of the eye can be increased by reducing the diameter of the pupil. Therefore, the use of muscarinic agonists (activators of the sphincter of the iris) or of alpha-2 agonists (relaxants of the dilator muscle of the iris) causes the pupil to contract and therefore increases the depth of visual focus.

The most common way of correcting presbyopia is the use of lenses; moreover, specific surgical treatments have been devised which include for example the insertion of intraocular lenses, lasers that reshape the cornea, and sclera expanders; finally, pharmacological treatments are known which are based on the use of pilocarpine, an analog of acetylcholine.

It is known that if pilocarpine is used as a single active ingredient in a topical composition, a percentage of pilocarpine greater than 0.5% by weight on the total weight of the composition is necessary in order to be effective in the treatment of presbyopia. It is also known that percentages of pilocarpine higher than 0.5% by weight are not tolerated, since they cause reddening of the eye, ocular and forehead pain and headaches; moreover, at the percentages of pilocarpine needed to improve the close-up reading ability of a long-sighted subject, the eye becomes so miotic as to cause a significant decrease in long-distance vision (Gilmartin et al., 1995, Ophthalmic and Physiological Optics, Pergamon Press, Oxford, GB, 15(5):475-479).

One known strategy for reducing the side effects of pilocarpine administered topically to the eye and at the same time maintain its effectiveness in presbyopia treatment is to combine it with alpha-1 and alpha-2 agents in order to utilize the synergistic effect with pilocarpine and consequently be able to reduce its percentage in the composition. Another strategy is to combine pilocarpine with non-steroid anti-inflammatory agents.

Patent application WO2008/075149 describes pilocarpine in combination with non-steroid anti-inflammatory agents, such as for example diclofenac, ketorolac, bromfenac, flurbiprofen, suprofen, pranoprofen, oxyphenbutazone, bendazac and indomethacin, in the treatment of visual impairments including presbyopia. The compositions exemplified therein contain at least pilocarpine HCl 1% and diclofenac sodium 0.5%. In some patients, pilocarpine at these doses causes diarrhea and dyspepsia.

Patent application WO2009/077736 describes pilocarpine in combination with alpha-2 agonists, such as for example brimonidine or iopidine, in the treatment of visual disorders including presbyopia. The compositions exemplified therein contain at least pilocarpine 0.25% and brimonidine 0.1%; at these percentages of the active ingredients there is eye discomfort and there are also side effects, such as for example reddening of part or all of the eye, caused by dilation of the blood vessels of the sclera.

Patent application WO2010/135731 describes pilocarpine in combination with alpha-2 agonists, such as for example brimonidine or naphazoline. These combinations are useful in the treatment of presbyopia. Examples are given of compositions containing at least pilocarpine 0.1% and brimonidine 0.1%, but results related to effectiveness in improving close-up visual acuity are shown only for combinations containing at least 0.25% pilocarpine and 0.2% brimonidine.

Patent application WO2013/041967 describes pilocarpine in combination with at least one of: alpha agonists or non-steroid anti-inflammatory agents having a selective Cox-2 inhibitory activity in the treatment of presbyopia. Among alpha agonists, mention is made for example of oxymethazoline, naphazoline, tetrahydrozoline, tramazoline and xylometazoline; among Cox-2 selective inhibitors, mention is made for example of: meloxicam, celecoxib, rofecoxib, valdecoxib, parecoxib, etoricoxib, nimesulide, etodolac and nabumetone. The compositions exemplified therein contain at least pilocarpine 1% and oxymethazoline 0.0125%, or pilocarpine 1% and meloxicam 0.015%.

Patent application WO2014/015183 describes pilocarpine in combination with alpha-1 agonists or antagonists and optionally a non-steroid anti-inflammatory agent. The alpha-1 agonists or antagonists are chosen among: phenylephrine, phenylpropanolamine, etylefrine, oxymetazoline, xilometazoline and tramazoline; the anti-inflammatory agents are chosen among: nepafenac, meloxicam, diclofenac, bendazac, ketorolac, oxyphenbutazone, bromfenac, flurbiprofen, pranoprofen, suprofen and indomethacin. The composition exemplified therein contains pilocarpine 0.247% and phenylephrine 0.78%.

### SUMMARY OF THE INVENTION

The Applicant has studied known formulations for the treatment of presbyopia in order to optimize these compositions in qualitative and quantitative terms and find effective formulations that might cause reduced discomfort and/or side effects.

The Applicant has found surprisingly a composition that is effective in the temporary treatment of presbyopia, characterized by the presence of active ingredients in low quantity that fall within specific percentage ranges.

This composition, in addition to ensuring effectiveness, has the additional advantage of reducing discomfort and/or the side effects caused by the individual active ingredients when administered alone or in combination topically to the eye at higher concentrations.

Therefore, a first object of the present invention is a composition comprising
(a) pilocarpine,
(b) at least one alpha-stimulant agonist
and/or (c) at least one non-steroid anti-inflammatory agent (NSAID)
wherein (a) is present in a percentage by weight lower than 0.40%, (b) and/or (c) is present in a percentage by weight lower than 0.090% based on the total volume of the composition.

A second object of the present invention is a composition as defined above, for use in the treatment of presbyopia.

A third object of the present invention is the use of a composition as defined above, in the preparation of a medicament for the treatment of presbyopia.

A fourth object of the present invention is a method for treating presbyopia in a subject, comprising the step of administering to said subject requiring treatment an effective quantity of a composition as defined above.

### Definitions

As used herein, the expression “to treat” means to improve or partially or completely reduce presbyopia. In the preferred embodiment, treatment of a subject means reducing presbyopia completely, ideally to the point of eliminating this condition. Partial improvement means that the degree of presbyopia is lower than what it would have been without treatment with the composition according to the invention. For example, the degree of presbyopia when using the treatment method according to the present invention can be at least 10%, 20%, 30%, 40%, 50%, 60%, 70%, 80%, 90%, 100% less than the degree of presbyopia that would have been present without treatment with the composition according to the present invention.

As used herein, the expression “administration of” means that the composition according to the invention has been placed in contact with the eye of the subject to be treated.

As used herein, the term “effective quantity” is the quantity of one or more active ingredients present in the composition of the present invention which, when administered to a subject with presbyopia, is effective in causing a miosis that is sufficient to treat the presbyopia so that close-up vision of the treated eye is restored partially or completely. A complete restoration of close-up vision must be sufficient to allow the subject to read the Times New Roman size 12 font without aid. A partial restoration of close-up vision must allow to see with less loss of focus. Therefore, an effective quantity refers to a quantity of the preparation that reduces presbyopia at least by 10%, 20%, 30%, 40%, 50%, 60%, 70%, 80%, 90%, 100%. In certain embodiments, the quantity of ophthalmic composition comprising (a) pilocarpine, (b) at least one alpha-stimulant agonist and/or (c) at least one nonsteroidal anti-inflammatory agent (NSAID) is effective in treating presbyopia for 12 hours, 11 hours, 10 hours, 9 hours, 8 hours, 7 hours, 6 hours, 5 hours, 4 hours, 3 hours, 2 hours or 1 hour. The degree of presbyopia can be measured by means of any method known in the art of ophthalmic exams.

As used herein, the expression “subject(s) with light presbyopia” indicates a subject whose vision could be corrected with lenses having diopters of 1.0 D or less, for example 0.75 D, 0.5 D.

As used herein, the expression “subject with severe presbyopia” indicates subjects whose vision could be corrected with lenses having diopters of more than approximately 1.0 D, for example 1.5 D, 2.0 D or more, for example up to 4.0 D in children.

As used herein, the term “OD” means right eye; “OS” means left eye.

As used herein, the expression “alpha-stimulant agonist” refers to compounds that preferentially stimulate alpha-1 and alpha-2 sympathetic nervous system receptors.

As used herein, the expression “pharmaceutically acceptable salt(s)” refers to a salt with inorganic acids, such as for example hydrochloric acid, hydrobromic acid, nitric acid, phosphoric acid, sulfuric acid and perchloric acid; or with organic acids such as acetic acid, oxalic acid, maleic acid, tartaric acid, citric acid, succinic acid or malonic acid. Other pharmaceutically acceptable salts include adipate, alginate, ascorbate, aspartate, benzene sulfonate, benzoate, disulfate, borate, butyrate, camphorate, camphor sulfonate, cyclopentane propionate, digluconate, dodecyl sulfate, ethane sulfonate, formate, fumarate, glucoheptonate, glycerophosphate, gluconate, hemisulfate, heptanoate, hexanoate, iodide, 2-hydroxyethane sulfonate, lactobionate, lactate, laurate, lauryl sulfate, malate, maleate, malonate, methane sulfonate, 2-naphthalene sulfonate, nicotinate, nitrate, oleate, oxalate, palmitate, palmoate, pectinate, persulfate, 3-phenylpropionate, phosphate, picrate, pivalate, propionate, stearate, succinate, sulfate, tartrate, thiocyanate, p-toluene sulfonate, undecanoate, valerate, and the like.

Representative salts of alkaline metals or alkaline earth metals include sodium, lithium, potassium, calcium, magnesium and the like. Additional pharmaceutically acceptable salts include non-toxic ammonium salts, quaternary ammonium, and amines.

### DETAILED DESCRIPTION OF THE INVENTION

The first object of the present invention is a composition comprising
(a) pilocarpine,
(b) at least one alpha-stimulant agonist
and/or (c) at least one non-steroid anti-inflammatory agent (NSAID)
wherein (a) is present in a percentage by weight lower than 0.40%, (b) and/or (c) is present in a percentage by weight lower than 0.090% referred to the total volume of the composition.

According to the first object of the invention, the active ingredients can be in the form of pharmaceutically acceptable salts; the percentage by weight of every active ingredient is referred to the non-salified active ingredient.

According to the first object of the invention, (b) is chosen among oxymethazoline, naphazoline, tetrahydrozoline, tramazoline, xylometazoline, iopidine and brimonidine and pharmaceutically acceptable salts thereof; preferably it is brimonidine.

According to the first object of the invention, (c) is chosen among nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac and nabumetone and pharmaceutically acceptable salts thereof; preferably it is ketorolac.

According to the first object of the invention, (a) is present in a percentage by weight preferably lower than 0.35%, lower than 0.30%; greater than 0.01%, preferably greater than 0.05%, greater than 0.08%; preferably comprised between 0.01 and 0.40% based on the total volume of the composition.

According to the first object of the invention, (b) and/or (c) is present in a percentage by weight preferably lower than 0.050%, lower than 0.030%, lower than 0.020%; greater than 0.001%, preferably greater than 0.002%, greater than 0.004%; preferably comprised between 0.001 and 0.090% based on the total volume of the composition.

In one embodiment, the composition according to the invention comprises (b) brimonidine and/or (c) ketorolac.

In a preferred embodiment, the composition according to the invention comprises (b) brimonidine, in a percentage by weight preferably lower than 0.030%, lower than 0.020%; greater than 0.001%, preferably greater than 0.002%, greater than 0.004%; preferably comprised between 0.001% and 0.030% based on the total volume of the composition.

In another preferred embodiment, the composition according to the invention comprises (c) ketorolac, in a percentage by weight preferably lower than 0.030%, lower than 0.020%; greater than 0.001%, preferably greater than 0.002%, greater than 0.004%; preferably comprised between 0.001% and 0.030% based on the total volume of the composition.

In a further preferred embodiment, the composition according to the invention comprises (b) brimonidine and/or ketorolac in a percentage by weight preferably lower than 0.030%, lower than 0.020%; greater than 0.001%, preferably greater than 0.002%, greater than 0.004%; preferably comprised between 0.001 and 0.030% based on the total volume of the composition.

In a further preferred embodiment, the composition according to the invention comprises
(a) pilocarpine in a percentage by weight comprised between 0.08% and 0.30% based on the total volume of the composition,
(b) brimonidine and/or (c) ketorolac in a percentage by weight comprised between 0.004% and 0.020% based on the total volume of the composition.

The compositions of the present invention can be prepared according to conventional methods known to the person skilled in the art; these compositions are preferably sterilized before use by means of conventional methods, by using membrane filters, autoclaves, etc.

According to the invention, the composition as described above comprises at least one ophthalmologically acceptable vehicle to provide the remainder up to 100% in volume of the composition.

According to the invention, the composition as described above is suitable for topical administration to the eye in the form of a solution, suspension, ointment, cream, gel or spray. Preferably, the composition of the present invention is in the form of a solution as eye drops.

The composition of the present invention can be prepared by using at least one ophthalmologically acceptable vehicle by mixing it with an effective quantity of active ingredients.

Acceptable vehicles for ophthalmic solutions are the ones commonly used and known to the person skilled in the art, such as for example water, aqueous mixtures and water miscible solvents, such as for example alkyl alcohols and arylalkyl alcohols, vegetable oils, polyalkylene glycols, base vaseline, ethylcellulose, ethyl oleate, carboxymethylcellulose, polyvinylpyrrolidone, isopropyl myristate.

The composition of the present invention can contain at least one ophthalmologically acceptable excipient.

Acceptable excipients for ophthalmic solutions are the ones commonly used and known to the person skilled in the art, such as for example emulsifiers, agents for imparting viscosity, solubilizing agents, mucoadhesive agents, agents for adjusting isotonicity, buffer agents to adjust the pH, preservatives, chelating agents, and the like.

Suitable emulsifiers include for example, but are not limited to, polyethylene glycol 200, 300, 400 and 600 and carbowax 1000, 1500, 4000, 6000 and 10000.

Suitable agents for imparting viscosity include for example, but are not limited to, non-ionic water-soluble polymers such as for example methylcellulose, carboxymethylcellulose, hydroxyethylcellulose, water-soluble cationic polymers such as for example Polyquat 37, fatty alcohols, fatty acids, anionic polymers, chondroitin, polyvinyl alcohol, and pullulan and/or salts thereof and/or mixtures thereof.

Suitable solubilizing agents include for example, but are not limited to, complex-forming agents, such as for example citric acid, ethylenediaminetetraacetate (EDTA), sodium metaphosphonate, succinic acid, urea, cyclodextrins, polyvinylpyrrolidone, diethylammonium orthobenzoate, Tweens and Spans, for example Tween 80; other solubilizing agents can be esters of polyoxyethylene sorbitan with fatty acids such as for example polysorbate 80, polyoxyethylene N-alkyl ethers, N-alkylamine N-oxides, poloxamers, organic solvents such as for example acetone, phospholipids and cyclodextrins.

Suitable mucoadhesive agents include for example, but are not limited to, macromolecules, polymers, oligomers or mixtures thereof that can adhere to the mucous membrane of the eye of the subject to be treated, such as for example carbopol, pectin, alginic acid, alginate, chitosan, hyaluronic acid, polysorbates such as for example polysorbate-20, -21, -40, -60, -61, -65, -80, -81, -85; polyethyleneglycols such as for example PEG-4, 6, -7, -8, -9, -10, -12, -14, -16, -18, -20, -32, -55, -90, -100, -135, -180, -240; oligosaccharides and polysaccharides such as for example polysaccharide of seeds of tamarind, gellan, carrageenan, xanthan gum, gum arabic, dextrane; esters and ethers of cellulose; polymers of modified cellulose, such as for example carboxymethylcellulose, hydroxyethylcellulose, hydroxypropylmethylcellulose; polyether polymers and oligomers, such as polyoxyethylene, condensation products of polyethylene oxide with fatty acids, fatty alcohols, fatty starches, polyhydric alcohols; polyether compounds as block copolymers of ethylene oxide and propylene oxide and mixtures thereof with other excipients such as for example polyvinyl alcohol; polyacrylamide, hydrolyzed polyacrylamide, polyvinylpyrrolidone, optionally cross-linked poly(meth)acrylic acid, such as for example carbomer.

Suitable agents for adjusting the composition to the desired range of isotonicity, also termed osmotic vectors, are known to the person skilled in the art and include, but are not limited to, glycerin; monosaccharides such as for example glucose and fructose; disaccharides such as for example maltotriose; polyalcohols such as for example mannitol and sorbitol; electrolytes such as for example sodium chloride, sodium hydrogen phosphate, potassium chloride, magnesium sulfate and calcium chloride. Suitable buffer agents are for example, but are not limited to, borate, carbonate, acetate, gluconate, citrate and phosphate.

Suitable preservatives are for example, but are not limited to, quaternary ammonium compounds, phenylmercuric salts, thimerosal, methyl and propyl parabens, benzyl alcohol, phenyl ethanol, benzalkonium chloride, benzyldodecinium bromide, stabilized oxygen-chlorine complexes (purite).

Suitable chelating agents for increasing the preservative effect are well-known to the person skilled in the art and include for example, but are not limited to, edetate salts.

Other conventional ingredients are for example amino acids such as for example glycin and alanine; vitamins and derivatives such as for example thiamine hydrochloride, riboflavin sodium phosphate, pyridoxine hydrochloride, nicotinamide, folic acid, biotin, vitamin A, L-ascorbic acid, and alpha-glycosil-L-ascorbic acid.

The composition of the present invention has a pH value in the range from 4.5 to 8.5. In some embodiments, the pH is comprised between 5 and 8. In other embodiments, the pH is comprised between 6 and 7.5. In other embodiments, the pH is approximately 7.3.

In a preferred embodiment, the composition according to the invention is further characterized by a specific percentage by weight of at least one osmotic vector, preferably sodium chloride, greater than 0.40% and lower than 0.50%, preferably 0.45% based on the total volume of the composition.

In a further even more preferred embodiment, the composition according to the invention comprises
(a) pilocarpine in a percentage by weight comprised between 0.08 and 0.30% on the total volume of the composition,
(b) brimonidine and/or (c) ketorolac in a percentage by weight comprised between 0.004 and 0.020% based on the total volume of the composition,
and at least one osmotic vector, preferably sodium chloride in a percentage by weight greater than 0.40% and lower than 0.50%, preferably 0.45% based on the total volume of the composition.

This even more preferred embodiment has the additional advantage of facilitating the rapid penetration of the active ingredients in the eye.

A second object of the present invention is a composition as defined above, for use in the treatment of presbyopia.

A third object of the present invention is the use of a composition as defined above, in the preparation of a medicament for treatment of presbyopia.

A fourth object of the present invention is a method for treating presbyopia in a subject, comprising the step of administering to said subject who needs said treatment an effective quantity of a composition as defined above.

According to the second and third object of the present invention, the composition can be suitable for use in the treatment of severe presbyopia and/or in the treatment of light presbyopia.

According to the fourth object of the present invention, the method of the present invention can be suitable for treatment of severe presbyopia and/or for treatment of light presbyopia.

The composition of the composition that is the object of the present invention is chosen according to the vision correction requirements or to the response of the subject to be treated.

For example, it is possible to administer to a subject with severe presbyopia, in general a subject with dark eyes and/or over 50 years of age, a composition comprising a higher percentage of pilocarpine.

Therefore, in a first embodiment of the method of the present invention, the percentage of pilocarpine administered is equal to or greater than 0.15%, preferably equal to or greater than 0.20%; and the percentage of at least one alpha-stimulant agonist and/or at least one non-steroid anti-inflammatory agent (NSAID) that is administered is at least 0.003%, more preferably at least 0.005%.

For example, it is possible to administer to a subject with a light form of presbyopia, in general a subject with light-colored eyes and/or an age comprised between 40 and 50 years, a composition comprising a smaller percentage of pilocarpine. Subjects who respond better to the treatment, such as for example young subjects (children), also can be administered a composition comprising a smaller percentage of pilocarpine.

Therefore, in a second embodiment of the method of the present invention, the percentage of pilocarpine that is administered is lower than 0.20%, preferably lower than 0.15%; and the percentage of at least one alpha-stimulant agonist and/or at least one non-steroid anti-inflammatory agent (NSAID) that is administered is at least 0.08%, more preferably at least 0.10%.

Therefore, according to the second, third and fourth object of the invention, the composition for treatment of severe presbyopia comprises
a percentage equal to or greater than 0.15%, preferably equal to or greater than 0.20%, of pilocarpine and
at least 0.003%, more preferably at least 0.005%, of an alpha-stimulant agonist and/or a non-steroid anti-inflammatory. agent (NSAID).

Therefore, according to the second, third or fourth object of the invention, the composition for treatment of light presbyopia comprises a percentage lower than 0.20%, preferably lower than 0.15%, of pilocarpine and at least 0.08%, more preferably at least 0.10%, of an alpha-stimulant agonist and/or a non-steroid anti-inflammatory agent (NSAID).

### Experimental Part

### EXAMPLE 1

A composition (1) representative of the invention was prepared, comprising
pilocarpine in a percentage of 0.225% by weight based on the total volume of the composition,
brimonidine in a percentage of 0.053% by weight based on the total volume of the composition,
ketorolac in a percentage of 0.017% by weight based on the total volume of the composition, artificial tears sufficient to reach 100% of the volume of the composition.

The artificial tears marketed under the name Protagent, containing polyvidone (polyvinylpyrrolidone) 20 mg, boric acid, sodium chloride, purified water, were used in the present composition.

As an alternative, it is possible to use other commercially available artificial tears, such as for example Hyalistil, containing hyaluronic acid sodium salt 2 mg, thiomersal; or Systane, containing polyethylene glycol 400, propylene glycol, hydroxypropyl-guar, boric acid, calcium chloride, magnesium chloride, potassium chloride, sodium chloride, zinc chloride, and polyquad (polidronium chloride 0.001%).

A variation (1a) that is representative of the invention was prepared, comprising (a) pilocarpine in a percentage of 0.225% by weight based on the total volume of the composition,
(b) brimonidine in a percentage of 0.053% by weight based on the total volume of the composition,
(c) ketorolac in a percentage of 0.017% by weight based on the total volume of the composition,
EDTA in a percentage of 0.1% by weight based on the total volume of the composition,
benzalkonium chloride in a percentage of 0.01% by weight based on the total volume of the composition, sodium chloride in a percentage of 0.45% by weight based on the total volume of the composition, sorbitol in a percentage of 0.06% by weight based on the total volume of the composition,
water to provide the remainder up to 100%.

The ingredients cited above were used according to known methods for the sterile preparation of ophthalmic solutions, adjusting the pH to 7.3, if necessary by using a buffer solution.

Compositions (1) and (1a) are particularly suitable for subjects with severe presbyopia, i.e., whose vision could be corrected with lenses having diopters of more than approximately 1.0 D, for example 1.5 D, 2.0 D or higher, for example up to 4.0 D in children.

### EXAMPLE 2

A composition (2) representative of the invention was prepared, comprising
(a) pilocarpine in a percentage of 0.105% by weight based on the total volume of the composition,
(b) brimonidine in a percentage of 0.011% by weight based on the total volume of the composition,
(c) ketorolac in a percentage of 0.017% by weight based on the total volume of the composition,
(d) artificial tears to provide the remainder up to 100% of the volume of the composition.

The artificial tears marketed under the name Protagent were used in the present composition.

A variation (2a) representative of the invention was prepared, comprising
(a) pilocarpine in a percentage of 0.105% by weight based on the total volume of the composition,
(b) brimonidine in a percentage of 0.011% by weight based on the total volume of the composition,
(c) ketorolac in a percentage of 0.017% by weight based on the total volume of the composition,
EDTA in a percentage of 0.1% by weight on the total volume of the composition, benzalkonium chloride in a percentage of 0.01% by weight based on the total volume of the composition, sodium chloride in a percentage of 0.45% by weight based on the total volume of the composition, sorbitol in a percentage of 0.06% by weight based on the total volume of the composition, water to provide the remainder up to 100%.

The ingredients cited above were used according to known methods for the sterile preparation of ophthalmic solutions, adjusting the pH to 7.3, if necessary by using a buffer solution.

Compositions (2) and (2a) are particularly suitable for subjects with light presbyopia, i.e., whose vision could be corrected with lenses having diopters lower than or equal to 1.0D, for example 0.75 D, 0.5 D.

### EXAMPLE 3

Visual acuity was measured by using the Jaeger scale.

The effect of composition (1) representative of the invention on the visual acuity of a group of 10 patients affected by severe presbyopia was assessed; these patients, without any kind of sight correction and prior to the treatment, had a visual acuity value, measured by means of an optotype chart for reading, comprised between J7 and J8.

The same subjects were administered a drop of composition (1) and their visual acuity was measured respectively after one hour, after 2 hours, after 4 hours, after 5 hours from treatment.

The results are presented in Table 1.

TABLE 1

| PATIENT | 1 HOUR | 2 HOURS | 4 HOURS | 5 HOURS |
|---|---|---|---|---|
| 1 | J3 | J3 | J3 | J5 |
| 2 | J3 | J3 | J3 | J3 |
| 3 | J5 | J3 | J3 | J5 |
| 4 | J5 | J4 | J3 | J3 |
| 5 | J3 | J3 | J3 | J3 |
| 6 | J3 | J3 | J5 | J5 |
| 7 | J3 | J3 | J3 | J3 |
| 8 | J5 | J3 | J3 | J5 |
| 9 | J3 | J3 | J3 | J3 |
| 10 | J3 | J4 | J3 | J3 |

As can be deduced from the data given in Table 1, the composition (1) representative of the invention, 1 hour after treatment, is capable of improving by at least three levels the Jaeger score with respect to the Jaeger score prior to treatment; in all the patients, the effect of the composition (1) representative of the invention occurred within the first hour of treatment and lasted beyond 5 hours after treatment.

At the recommended dose of one drop no more than 2 times per day approximately 6-7 hours apart, only 2 of the 10 treated patients reported a slight reddening of the eyes and a mild headache in the first days of administration. None of the patients reported difficulty in light perception, obfuscated long-range vision, itching or other manifestation of allergic reaction, diarrhea or dyspepsia.

### EXAMPLE 4

Visual acuity was measured by using the Jaeger scale.

The effect of composition (2) representative of the invention on the visual acuity of a group of 10 patients affected by light presbyopia was evaluated; these patients, without any type of visual correction and prior to treatment, had a visual acuity value, measured by means of an optotype chart for reading, comprised between J5 and J6.

The same subjects were administered a drop of composition (2) and their visual acuity was measured respectively after one hour, after 2 hours, after 4 hours, after 5 hours from treatment.

The results are presented in Table 2.

TABLE 2

| PATIENT | 1 HOUR | 2 HOURS | 4 HOURS | 5 HOURS |
|---|---|---|---|---|
| 1 | J3 | J3 | J3 | J5 |
| 2 | J3 | J3 | J3 | J3 |
| 3 | J5 | J3 | J3 | J5 |
| 4 | J5 | J4 | J3 | J3 |
| 5 | J3 | J3 | J3 | J3 |
| 6 | J3 | J3 | J5 | J5 |
| 7 | J3 | J3 | J3 | J3 |
| 8 | J5 | J3 | J3 | J5 |
| 9 | J3 | J3 | J3 | J3 |
| 10 | J3 | J4 | J3 | J3 |

As can be deduced from the data provided in Table 2, the composition (2) representative of the invention, 1 hour after treatment, is capable of improving by at least three levels the Jaeger score with respect to the Jaeger score prior to treatment; in all the patients, the effect of the composition (2) representative of the invention occurred within the first hour of treatment and lasted beyond 5 hours after treatment.

At the recommended dose of one drop no more than 2 times per day approximately 6-7 hours apart, none of the 10 treated patients reported reddening of the eyes and headache, even in the first days of administration. Moreover, none of the treated patients reported difficulty in light perception, obfuscated long-distance vision, itching or other manifestation of allergic reaction, diarrhea, or dyspepsia.

1. A composition comprising:
pilocarpine; and
at least one of: an alpha-stimulant agonist and a nonsteroidal anti-inflammatory agent (NSAID);
wherein the pilocarpine is present in a percentage by weight less than 0.40%; and
wherein each one of the alpha-stimulant agonist, if present, and the NSAID, if present, is less than 0.090 percent by weight based on a total volume of the composition.

2. The composition according to claim 1, wherein the alpha-stimulant agonist is at least one of: oxymethazoline, naphazoline, tetrahydrozoline, tramazoline, xylometazoline, iopidine and brimonidine.

3. The composition according to claim 1, wherein the NSAID is at least one of: nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac and nabumetone.

4. The composition according to claim 1, wherein the pilocarpine is present between about 0.01 and about 0.40 percent by weight based on the total volume of the composition.

5. The composition of claim 1, wherein the alpha-stimulant agonist, if present, and the NSAID, if present is between about 0.001 and about percent by weight of the total volume of the composition; or wherein the sum of the alpha-stimulant agonist and the NSAID is between about 0.001 and about 0.090 percent by weight of the total volume of the composition.

6. The composition according to claim 1, wherein:
(a) pilocarpine is between about 0.08 and about 0.30 percent by weight of the total volume of the composition; and
(b) the alpha-stimulant agonist, if present, is brimonidine and the NSAID, if present, is ketorolac, and each of brimonidine and ketorolac, or the sum of brimonidine and ketorolac constitutes between about 0.004 and about 0.020 of the total volume of the composition.

7. The composition according to claim 1, wherein:
(a) pilocarpine is equal to or greater than 0.15 percent by weight of the total volume of the composition; and
(b) the alpha-stimulant agonist, if present, is brimonidine and the NSAID, if present, is ketorolac, and each of brimonidine and ketorolac, or the sum of brimonidine and ketorolac is present in a concentration of at least 0.003 percent by weight of the total volume of the composition; or
(a) pilocarpine is less than 0.20 percent by weight of the total volume of the composition; and
(b) the alpha-stimulant agonist, if present, is brimonidine and the NSAID, if present, is ketorolac, and each of brimonidine and ketorolac, or the sum of brimonidine and ketorolac is present in a concentration of at least 0.08 percent by weight of the total volume of the composition.

8. The composition according to claim 1, further comprising at least one ophthalmologically acceptable vehicle.

9. The composition according to claim 8, constituted as a solution, suspension, ointment, cream, gel or spray.

10. The composition according to claim 1, adapted for use in the treatment of presbyopia.

11. The composition according to claim 2, wherein the NSAID is at least one of:
nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac and nabumetone.

12. The composition according to claim 1, wherein the amount of pilocarpine is:
less than 0.35 percent by weight;
less than 0.30 percent by weight;
greater than 0.01 percent by weight;
greater than 0.05 percent by weight; or
greater than 0.08 percent by weight
of the total volume of the composition.

13. The composition according to claim 1, wherein the amount of alpha-stimulant agonist, if present, and the amount of NSAID, if present, or the sum of the amounts of alpha-stimulant agonist and NSAID is:
less than 0.050 percent by weight;
less than 0.030 percent by weight;
less than 0.020 percent by weight;
greater than 0.001 percent by weight;
greater than 0.002 percent by weight; or
greater than 0.004 percent by weight of the total volume of the composition.

14. The composition according to claim 1, wherein:
(a) the amount of pilocarpine is equal to or greater than 0.20 percent by weight of the total volume of the composition; and
(b) the alpha-stimulant agonist, if present, is brimonidine and the NSAID, if present, is ketorolac and each of brimonidine and ketorolac, or the sum of brimonidine and ketorolac is in a concentration of at least 0.005 percent by weight of the total volume of the composition;
or
(a) pilocarpine is less than 0.15 percent by weight of the total volume of the composition; and
(b) the alpha-stimulant agonist, if present, is brimonidine and the NSAID, if present, is ketorolac, and each of brimonidine and ketorolac or the sum of brimonidine and ketorolac is present in a concentration of at least 0.080 percent by weight of the total volume of the composition.

15. The composition according to claim 8, wherein the ophthalmologically acceptable vehicle is at least one osmotic vector.

16. The composition according to claim 15, wherein said osmotic vector is sodium chloride.

17. The composition according to claim 15, wherein the osmotic vector constitutes between about 0.40 and about 0.50 percent by weight of the total volume of the composition or wherein the osmotic vector constitutes about 0.45 percent by weight of the total volume of the composition.

18. The composition according to claim 8, wherein the composition is adapted for topical administration into the eye of a patient.

19. The composition according to claim 1, further comprising an ophthalmologically acceptable excipient selected from the group consisting of:
an emulsifier;
an agent for controlling viscosity;
a solubilizing agent;
a mucoadhesive agent;
an agent for controlling isotonicity;
a buffer agent for controlling pH;
a preservative; and
a chelating agent.

20. A method for treating presbyopia in a subject, comprising the step of administering to said subject requiring treatment an effective quantity of a composition as defined in claim 1.
